(19) United States
(12) Patent Application Publication
(10) Pub. No.: US 2005/0188478 A1
(43) Pub. Date: Sep. 1, 2005

(54) Composition for dyeing keratin fibers, comprising at least one alcohol oxidase and at least one sugar-based nonionic surfactant, and a process using this composition

(76) Inventor: Gregory Plos, Tokyo (JP)

Correspondence Address: Thomas L. Irving;FINNEGAN, HENDERSON, FARABOW,, GARRETT & DUNNER, L.L.P., 901 New York Avenue, N.W., Washington, DC, 20001-4413, US

(21) Appl. No.: 11/044,274

(22) Filed: Jan. 28, 2005

Related U.S. Application Data
(60) Provisional application No. 60/545,481, filed on Feb. 19, 2004.

(30) Foreign Application Priority Data
Jan. 28, 2004 (FR) 0400780

(51) Int. Cl. A61K 7/13

(52) U.S. Cl. 8/405

(57) ABSTRACT

The disclosure relates to a composition for dyeing keratin fibers, for example, human keratin fibers such as the hair, comprising, in a medium that is suitable for dyeing, at least one oxidation dye precursor, at least one alcohol oxidase enzyme, at least one substrate for said enzyme and at least one surfactant. The present disclosure further relates to a process for dyeing keratin fibers using the compositions disclosed herein and also to a dyeing “kit”.

This application claims benefit of U.S. Provisional Application No. 60/545,481, filed Feb. 19, 2004 and French Patent Application No. 0400780 filed Jan. 28, 2004 and which are herein incorporated by reference.

The present disclosure relates to a composition for dyeing keratin fibers, for example, human keratin fibers such as the hair, comprising, in a medium that is suitable for dyeing, at least one oxidation dye precursor, at least one alcohol oxidase enzyme, at least one substrate for said enzyme and at least one nonionic surfactant.

It is known practice to dye keratin fibers, for example, human hair, with dye compositions comprising oxidation dye precursors, for example, ortho- or para-phenylenediamines, ortho- or para-aminophenols, and heterocyclic compounds, which are generally referred to as oxidation bases. These oxidation bases, are colourless or weakly coloured compounds which, when combined with oxidizing products, may give rise to coloured compounds by a process of oxidative condensation.

It is also known that the shades obtained with these oxidation bases may be varied by combining them with couplers or coloration modifiers, the latter is chosen from aromatic meta-diamines, meta-aminophenols, meta-diphenols and certain heterocyclic compounds such as indole compounds.

The variety of molecules used as oxidation bases and couplers makes it possible to obtain a wide range of colours.

The “permanent” coloration obtained by means of these oxidation dyes often satisfies a certain number of requirements. It may have no toxicological drawbacks and it may allow shades of the desired intensity to be obtained and have good resistance to external agents (light, bad weather, washing, permanent waving, perspiration and rubbing).

The dyes may also allow white hairs to be covered and, lastly, they may be as unselective as possible, that is to say that they may allow the smallest possible differences in coloration to be produced over the entire length of the same keratin fiber, which is generally differently sensitized (i.e. damaged) between its end and its root.

The dyeing is generally performed in strongly alkaline medium, in the presence of hydrogen peroxide. However, the use of alkaline media in the presence of hydrogen peroxide may have the drawback of causing considerable degradation of the fibers, and also bleaching of keratin fibers, which is not always desirable.

The oxidation dyeing of keratin fibers may also be performed using oxidizing systems other than hydrogen peroxide, such as enzymatic systems. Thus, French Patent Application No. FR 2 769 219 describes the use of a uricase enzyme and of its uric acid substrate in oxidation dyeing to dye keratin fibers. European Patent Application No. EP-A-0 310 675 describes the use of oxidation dye precursors of benzenic type in combination with enzymes such as pyranose oxidase and glucose oxidase. More recently, French Patent Application No. FR 2 833 492 describes the use of alcohol oxidase enzyme as sole enzyme in an oxidation dye composition for dyeing keratin fibers.

Disclosed herein in one embodiment are novel homogeneous and stable compositions for dyeing keratin fibers by oxidation dyeing, using an oxidizing system other than hydrogen peroxide.

The inventor has discovered, unexpectedly, that it may be possible to achieve this aim by using at least one oxidation dye precursor, at least one alcohol oxidase enzyme, at least one substrate for said enzyme and at least one sugar-based nonionic surfactant in a composition for dyeing keratin fibers, for example, human keratin fibers such as the hair.

In one embodiment, the composition according to the disclosure may make it possible to obtain homogeneous formulations which, once applied, respect the nature of the keratin fibers and do not have the solubilization and crystallization problems encountered with the uric acid/uricase system. In one embodiment, the inventor has also noted that the stability of the composition according to the disclosure, for example, the stability of the alcohol oxidase enzyme of this composition, is improved.

In one embodiment, the compositions according to the present disclosure may have the advantage that they lead to the production of dyeing results with strong, unselective and fast colours, and these compositions are capable of generating varied shades of intense and uniform colour, without any significant degradation of the hair. In addition, it has been noted that the use of such a composition may improve the hold of permanent-waved hair and reduces the porosity of the hair.

Other characteristics, aspects, subjects and advantages of the present disclosure will emerge even more clearly on reading the description and the concrete, but non-limiting, examples that follow.

The sugar-based nonionic surfactant that may be used according to the present disclosure may be chosen from:
1—alkylpolyglucosides;
2—fatty acid esters of sugar or of an alkyl sugar;
3—fatty sucramides; and
4—mixtures thereof.

The alkylpolyglucosides, (1), may be chosen from formula (I):
R₁—O—(R₂—O)ₐ—(L)_{b} (I)
wherein:
R¹ is chosen from linear and branched alkyl and alkenyl groups comprising from 8 to 24 carbon atoms; and alkylphenyl groups wherein the linear or branched alkyl group ranges from 8 to 24 carbon atoms;
R² is chosen from alkylene groups comprising from 2 to 4 carbon atoms;
L is chosen from a reduced sugar comprising from 5 or 6 carbon atoms;
a is a number ranging from 0 to 10; and
b is a number ranging from 1 to 15.

Alkylpolyglucosides may be, for example, compounds of formula (I) wherein R¹ is chosen from linear and branched alkyl and/or alkenyl groups comprising from 9 to 14 carbon atoms, a is a number ranging from 0 to 3, for example, 0 and L is chosen from glucose, fructose and galactose. The degree of polymerization (S) of the saccharide, i.e. the value of b in formula (I), may, for example, range from 1 to 15. Reduced sugars comprising 80%, or more, of sugars whose degree of polymerization (S) takes a value ranging from 1 to 4 may, for example, be used.

Compounds of formula (I) may be, for example, chosen from the products sold by the company Henkel under the name APG, such as the products APG 300, APG 350, APG 500, APG 550, APG 625 and APG Base 10-12; the products sold by the company SEPPIC under the names Triton CG 110 (or Oramix CG 110) and Triton CG 312 (or Oramix NS 10); those sold by the company BASF under the name Lutensol GD 70; those sold by the company Henkel under the names Plantaren 1200, Plantaren 1300 and Plantaren 2000, and Plantacare 2000, Plantacare 818 and Plantacare 1200.

The fatty acid ester of a sugar or of an alkyl-sugars, (2), which may be used in the present disclosure may be chosen from esters of a C₄-C₂₂ fatty acid and of a sugar or of an alkyl-sugar, for example:
(2)(a)(C₁-C₄)alkylglucoside esters, such as:
   methylglucoside monostearate, for instance the product sold under the name Grillocose IS by the company Grillowerke;
   methylglucoside sesquistearate, for instance the product sold under the name Glucate SS by the company Amerchol;
   ethyl-6-glucoside decanoate, for instance the product sold under the name Biosurf 10 by the company Novo;
   the mixture (82/7) of ethyl-6-glucoside mono- and dicocoate, for instance the product sold under the name Biosurf Coco by the company Novo;
   the mixture (84/8) of ethyl-6-glucoside mono- and dilaurate, for instance the product sold under the name Biosurf 12 by the company Novo; and
   C₁₂-C₁₈ fatty acid monoesters of butylglucoside, such as butylglucoside monococoate, for instance the product sold under the name Rewopol V3101 or Rewosan V3101, and butylglucoside monococoate polyoxyethylenated with 3 mol of ethylene oxide, for instance the product sold under the name Rewopol V3122 by the company Rewo.
(2)(b) glucose esters such as:
   O-hexadecanoyl-6-αD-glucose, O-octanoyl-6-D-glucose, O-oleyl-6-D-glucose and O-linoleyl-6-D-glucose, which are known compounds that may be prepared, for example, from the corresponding acid chloride and from D-glucose, according to the method described by E. Reinefeld et al.; “Die Stärke” No.6-pages 181-189,-1968.
(2)(c) sucrose monoesters, such as:
   sucrose monolaurate, for instance the product sold under the name Grilloten LES 65, and
   the sucrose monococoate sold under the name Grilloten LES 65K by the company Grillo-Werke.

The fatty sucramides, (3), which may be used in the present disclosure are compounds comprising at least one amide function and including at least one sugar or sugar-based portion and at least one fatty chain; such compounds may result, for example, from the action of a fatty acid or of a fatty acid derivative on the amine function of an amino sugar, or from the action of a fatty amine on a sugar comprising a carboxylic acid function (in free form or in lactone form) or carboxylic acid derivative or a carbonyl function, optionally in the presence of suitable core agents.

The fatty sucramides (3) used may, for example, be chosen from:
(3)(a) N-substituted aldonamides, and
(3)(b) polyhydroxylated fatty acid amides or mixtures thereof.

(3)(a) The N-substituted aldonamides that may be used according to the disclosure may be chosen from those described in European Patent Application No. EP-A-550 106, the content of which is herein incorporated by reference.

Among these, non-limiting mention may be made of:
N-substituted lactobionamides, N-substituted maltobionamides, N-substituted cellobionamides, N-substituted mellibionamides and N-substituted gentiobionamides such as:
   (i) N-alkyl lactobionamides, N-alkyl maltobionamides, N-alkyl cellobionamides, N-alkylmellibionamides or N-alkyl gentiobionamides mono- or disubstituted with groups chosen from linear and branched, saturated and unsaturated aliphatic hydrocarbon-based groups, optionally comprising hetero atoms comprising from 1 to 36 carbon atoms, for example, from 1 to 24 carbon atoms and further, for example, from 8 to 18 carbon atoms (for example methyl, ethyl, amyl, hexyl, heptyl, nonyl, decyl, undecyl, dodecyl, tridecyl, tetradecyl, pentadecyl, hexadecyl, heptadecyl, octadecyl; allyl, undecenyl, oleyl, linoleyl, propenyl or heptenyl), with an aromatic hydrocarbon-based group (for example benzyl, anilino, substituted benzyl, phenylethyl, phenoxyethyl or vinylbenzyl) or cycloaliphatic groups (for example cyclopentyl or cyclohexyl);
   (ii) N-lactobionyl amino acid esters, wherein the amino acid may be, for example, chosen from alanine, valine, glycine, lysine, leucine, arginine, aspartic acid, glutamic acid, threonine, serine, cysteine, histidine, tyrosine or methionine or may be chosen, for example, from β-alanine, sarcosine, γ-aminobutyric acid, ornithine, citrulline, or equivalents thereof; said N-lactobionyl amino acid esters is monosubstituted with a group of formula (II): 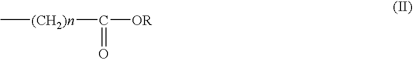
      wherein:
R is chosen from aliphatic hydrocarbon-based groups comprising from 1 to 36 carbon atoms; and
n is an integer greater than 1; and also the corresponding N-maltobionyl amino acid esters, N-mellibionyl amino acid esters, N-cellobionyl amino acid esters and N-gentiobionyl amino acid esters,
   (iii) N-(alkyloxy)alkyl-lactobionamides mono- or disubstituted with a group —(CH₂)ₙOR′ wherein R′ is an aliphatic, aromatic or cycloaliphatic hydrocarbon-based group as defined in paragraph (i);
   (iv) N-(polyalkyloxy)alkyl lactobionamides, N-(polyalkyloxy)alkyl maltobionamides, N-(polyalkyloxy)alkyl cellobionamides, N-(polyalkyloxy)alkyl mellibionamides or N-(polyalkyloxy)alkyl gentiobionamides, which are mono- or disubstituted with a group
      —R₁—(OR₁)ₙR₁R₂
      wherein R₁ is an alkylene group such as ethylene or propylene, or mixtures thereof, n is an integer greater than 1 and R₂ is a lactobionamide, maltobionamide, cellobionamide, melliobionamide or gentiobionamide group.

(3)(b) The polyhydroxylated fatty amides in accordance with the present disclosure may, for example, be chosen from those described in European Patent No. EP-B-550 656, the content of which forms an integral part of the description, and which correspond to formula (III): 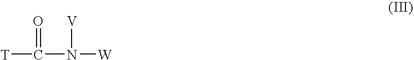
wherein:
T is chosen from C₅-C₃₁ hydrocarbon-based groups, for example, linear C₇-C₁₅ alkyl or alkenyl chains, or mixtures thereof;
V is chosen from a hydrogen atom, a C₁-C₄ hydrocarbon-based group, a 2-hydroxyethyl or 2-hydroxypropyl group, or mixtures thereof, for example, a C₁-C₄ alkyl such as methyl, ethyl, propyl, isopropyl or N-butyl and further, for example, methyl;
W is chosen from a polyhydroxy hydrocarbon-based group with a linear hydrocarbon-based chain comprising at least 3 hydroxyl groups directly linked to the chain, or an alkoxylated derivative of said group, for example, ethoxylated or propoxylated;
W may be chosen from, for example, a reducing sugar derivative obtained via a reductive amination reaction, for example, a glycityl group. Among the reducing sugars that may be mentioned are glucose, maltose, lactose, galactose, mannose and xylose.

W may, for example, also be chosen from:
—CH₂—(CHOH)ₙ—CH₂OH;
—CH—(CH₂OH)—(CHOH)ₙ₋₁—CH₂OH; and
—CH₂—(CHOH)₂(CHOR′)(CHOH)—CH₂OH
wherein n is an integer ranging from 3 to 5 and R′ is hydrogen or a cyclic or aliphatic monosaccharide, or an alkoxylated derivative thereof,
for example, a glycityl group wherein n is equal to 4, for example, a —CH₂—(CHOH)₄—CH₂OH group.

The group T-CON=may be, for example, cocamide, stearamide, oleamide, lauramide, myristyramide, capricamide, palmitamide or tallow amide.

According to the present disclosure, the alkylpolyglucosides (1) are, for example, used as sugar-based nonionic surfactants.

The sugar-based nonionic surfactant may be present in an amount ranging from 0.05% to 30% by weight, relative to the total weight of the dye composition, for example, from 0.1 % to 15% by weight, relative to the total weight of the dye composition.

In the context of the present disclosure, the alcohol oxidase enzymes used in the dye composition in accordance with the disclosure belong to the class E.C.1.1.3 of the enzyme nomenclature (see Enzyme Nomenclature, Academic Press Inc; 1992).

Said enzymes may be chosen from primary alcohol oxidases (EC1.1.3.13), secondary alcohol oxidases (EC 1.1.3.18), long-hydrocarbon-chain alcohol oxidases (EC 1.1.3.20), polyvinyl alcohol oxidases (EC 1.1.3.30), vanillyl alcohol oxidase (EC 1.1.3.38) and aromatic alcohol oxidases (EC 1.1.3.7), also known as aryl alcohol oxidases.

Alcohol oxidase enzymes form a class of 2-electron oxidoreductase enzymes.

The alcohol oxidase enzyme used in the dye composition according to the disclosure may be derived from an extract of plants, of animals, of micro-organisms (bacterium, fungus, yeast, microalga or virus), of differentiated or undifferentiated cells, obtained in vivo or in vitro, unmodified or genetically modified, or synthetic (obtained by chemical or biotechnological synthesis).

By way of non-limiting example, the alcohol oxidase enzyme may be derived from one of the following species: Rhodococcus erythropolis, Pseudomonas pseudoalcaligenes which are bacteria, Aspergillus niger, Kamagataella pastoris, Phanerochaete chrysosporium, Polyporus obtusus, Hansenula polymorpha, Poria contigua, Penicillium simplicissimum, Pleurotus pulmonarius (fungi), Pichia sp. (pastoris, methanolica, angusta) and Candida sp. (boidinii, albicans, tropicalis) (yeasts), Pinus strobus, which is a species of plant origin, Gastropode mollusc and Manduca sexta, which are of animal origin.

The enzyme used in the composition may be, for example, an oxidase alcohol derived from Pichia pastoris.

Generally, the concentration of alcohol oxidase enzyme used in the dye composition may range from 0.05% to 20% by weight, relative to the total weight of the composition, for example, from 0.1% to 10% by weight and further, for example, from 0.5% to 8% by weight, relative to the total weight of the composition.

The enzymatic activity of the alcohol oxidase enzymes used in accordance with the disclosure may be defined from the oxidation of the donor under aerobic conditions. One unit U corresponds to the amount of enzyme leading to the generation of 1 μmol of hydrogen peroxide per minute at a given pH and at a temperature of 25° C.

The amount of alcohol oxidase may, for example, be present in an amount ranging from 10³ U to 10⁵ U per 100 g of dye composition, for example, from 2×10³ U to 5×10⁴ U per 100 g of dye composition.

The substrate(s) for said enzyme in the compositions of the disclosure are also known as donors for the enzyme. The substrate for the enzyme may be, for example, an alcohol chosen from primary and secondary alcohols, alcohols with a long hydrocarbon-based chain and aromatic alcohols. As non-limiting examples of donors for the primary alcohol oxidases, mention may be made of primary alcohols comprising from 1 to 6 carbon atoms; as donors for the aryl alcohol oxidases: benzyl alcohol, 4-tert-butylbenzyl alcohol, 3-hydroxy-4-methoxybenzyl alcohol, veratryl alcohol, 4-methoxybenzyl alcohol and cinnamyl alcohol; 2,4-hexadien-1-ol may also be used as donor for the aryl alcohol oxidases.

According to another variant of the disclosure, the substrate for the enzyme is a compound bearing at least one aliphatic or aromatic alcohol function, which is suitable for reacting with the enzyme used. The compound bearing at least one aliphatic or aromatic alcohol function may be an oxidation dye precursor or a cosmetically acceptable adjuvant, for example a polymer, a surfactant or a preserving agent bearing at least one alcohol function. Further, for example, the substrate for the enzyme may be an oxidation dye precursor bearing at least one aliphatic or aromatic alcohol function. For example, N-(β-hydroxypropyl)-para-phenylenediamine, which bears a primary alcohol function, may serve as oxidation base and as substrate for the alcohol oxidase. Similarly, oxidation dyes, such as meta- or para-aminophenol, may fulfil both functions. Such precursors are described hereinbelow. In this variant, the use of other substrates for the enzyme is optional.

The present disclosure is directed towards a composition for dyeing keratin fibers, for example, human keratin fibers such as the hair, comprising, in a medium that is suitable for dyeing, at least one oxidation dye precursor; at least one alcohol oxidase enzyme; at least one substrate, bearing an alcohol function, for the at least one alcohol oxidase enzyme, and at least one sugar-based nonionic surfactant, the at least one substrate for the at least one alcohol oxidase enzyme optionally substituted (i.e. replaced) totally or partially with the oxidation dye precursor in the case where at least one substrate for the at least one alcohol oxidase enzyme bears at least one aliphatic or aromatic alcohol function.

The use of the composition in accordance with the disclosure may reduce the risks associated with the handling of hydrogen peroxide. Furthermore, the concentration of preserving agents in the compositions according to the present disclosure may be reduced by supplying compounds comprising an alcohol function which also have preserving properties.

The substrate concentration for the enzyme may, for example, range from 0.01% to 60% by weight, relative to the total weight of the composition, for example, from 0.05% to 30% by weight, relative to the total weight of the composition.

The standard oxidation bases may, for example, be chosen from para-phenylenediamines, bis(phenyl)alkylenediamines, para-aminophenols, ortho-aminophenols and heterocyclic bases, and the addition salts thereof.

The para-phenylenediamines may, for example, be chosen from para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-amino-N,N-bis(β-hydroxyethyl)-2-methylaniline, 4-amino-2-chloro-N,N-bis(β-hydroxyethyl)aniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N,N-(ethyl-β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′aminophenyl)pyrrolidine, and the addition salts thereof with an acid.

The para-phenylenediamines may, by further example, be chosen from para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine and the addition salts thereof with an acid.

Among the bis(phenyl)alkylenediamines, non-limiting mention may be made, by way of example, of N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetra-methylenediamine, N,N′-bis(ethyl)-N,N′-bis(4′-amino-3′-methylphenyl)ethylenediamine and 1,8-bis(2,5-diaminophenoxy)-3,6-dioxaoctane, and the addition salts thereof with an acid.

Among the para-aminophenols, non-limiting mention may be made, by way of example, of para-aminophenol, 4-amino-3-methylphenol, 4-amino-3-fluorophenol, 4-amino-3-chlorophenol, 4-amino-3-hydroxymethylphenol, 4-amino-2-methylphenol, 4-amino-2-hydroxymethylphenol, 4-amino-2-methoxymethyl phenol, 4-amino-2-aminomethylphenol, 4-amino-2-(□-hydroxyethylaminomethyl)phenol, 4-amino-2-fluorophenol, 4-amino-2,6-dichlorophenol, 4-amino-6[(5′-amino-2′-hydroxy-3′-methyl)phenylmethyl]-2-methylphenol and bis-(5′-amino-2′-hydroxyl)phenylmethane, and the addition salts thereof with an acid.

Among the ortho-aminophenols, non-limiting mention may be made, by way of example, of 2-aminophenol, 2-amino-5-methylphenol, 2-amino-6-methylphenol and 5-acetamido-2-aminophenol, and the addition salts thereof with an acid.

Among the heterocyclic bases, non-limiting mention may be made, by way of example, of pyridine derivatives, pyrimidine derivatives and pyrazole derivatives.

Among the pyridine derivatives, non-limiting mention may be made of the compounds described, for example, in UK Patent Nos. GB 1 026 978 and GB 1 153 196, such as 2,5-diaminopyridine, 2-(4-methoxyphenyl)amino-3-aminopyridine, and 3,4-diaminopyridine, and the addition salts thereof with an acid.

Other pyridine oxidation bases that are useful in the present disclosure may be chosen from the 3-aminopyrazolo[1,5-a]pyridine oxidation bases or the addition salts thereof described, for example, in French Patent Application FR 2 801 308. By way of example, non-limiting mention may be made of pyrazolo[1,5-a]pyrid-3-ylamine; 2-acetylaminopyrazolo[1,5-a]pyrid-3-ylamine; 2-morpholin-4-ylpyrazolo[1,5-a]pyrid-3-ylamine; 3-aminopyrazolo[1,5-a]pyridine-2-carboxylic acid; 2-methoxypyrazolo[1,5-a]pyrid-3-ylamine; (3-aminopyrazolo[1,5-a]pyrid-7-yl)methanol; 2-(3-aminopyrazolo[1,5-a]pyrid-5-yl)ethanol; 2-(3-aminopyrazolo[1,5-a]pyrid-7-yl )ethanol; (3-aminopyrazolo[1,5-a]pyrid-2-yl)-methanol; 3,6-diaminopyrazolo[1,5-a]pyridine; 3,4-diaminopyrazolo[1,5-a]pyridine; pyrazolo[1,5-a]pyridine-3,7-diamine; 7-morpholin-4-ylpyrazolo[1,5-a]pyrid-3-ylamine; pyrazolo[1,5-a]pyridine-3,5-diamine; 5-morpholin-4-ylpyrazolo[1,5-a]pyrid-3-ylamine; 2-[(3-aminopyrazolo[1,5-a]pyrid-5-yl)(2-hydroxyethyl)amino]ethanol; 2-[(3-aminopyraz-olo[1,5-a]pyrid-7-yl )(2-hydroxyethyl)amino]ethanol; 3-aminopyrazolo[1,5-a]pyrid-5-ol; 3-aminopyrazolo[1,5-a]pyrid-4-ol; 3-aminopyrazolo[1,5-a]pyrid-6-ol; 3-aminopyrazolo[1,5-a]-pyrid-7-ol; and also the addition salts thereof with an acid.

Among the pyrimidine derivatives, non-limiting mention may be made of the compounds described, for example, in German Patent No. DE 2 359 399; Japanese Patent Nos. JP 88-169 571 and JP 05-163 124; European Patent No. EP 0 770 375 or International Patent Application No. WO 96/15765, such as 2,4,5,6-tetra-aminopyrimidine, 4-hydroxy-2,5,6-triaminopyrimidine, 2-hydroxy-4,5,6-triaminopyrimidine, 2,4-dihydroxy-5,6-diaminopyrimidine and 2,5,6-triaminopyrimidine and the addition salts thereof and the tautomeric forms thereof, when a tautomeric equilibrium exists.

Among the pyrazole derivatives, non-limiting mention may be made of the compounds described in German Patent Nos. DE 3 843 892 and DE 4 133 957 and International Patent Application Nos. WO 94/08969 and WO 94/08970, French Patent Application No. FR-A-2,733,749 and German Patent Application No. DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl )pyrazole, 4,5-diamino-1,3-dimethyl pyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methyl pyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole and 3,5-diamino4-(β-hydroxyethyl)amino-1-methylpyrazole, and the addition salts thereof.

For example, the concentration of the oxidation base may range from 0.0001% to 20%, for example, from 0.005% to 6% by weight, relative to the total weight of the composition.

Oxidation couplers may, for example, be chosen from meta-phenylenediamines, meta-aminophenols, meta-diphenols, naphthalenic couplers and heterocyclic couplers, and the addition salts thereof.

Non-limiting examples that may be mentioned may be chosen from 2-methyl-5-aminophenol, 5-N-(β-hydroxyethyl)amino-2-methylphenol, 6-chloro-2-methyl-5-aminophenol, 3-aminophenol, 1,3-dihydroxybenzene (or resorcinol), 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylami nobenzene, sesamol, 1-β-hydroxyethylamino-3,4-methylened ioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene and 2,6-bis(β-hydroxyethylamino)toluene, and the addition salts thereof.

For example, the concentration of the oxidation coupler may range from 0.0001% to 20% by weight, for example, from 0.005% to 6% by weight, relative to the total weight of the composition.

In general, the addition salts with an acid that may be used for the oxidation bases and couplers may be chosen from the hydrochlorides, hydrobromides, sulfates, citrates, succinates, tartrates, lactates, tosylates, benzenesulphonates, phosphates and acetates.

The addition salts that may be used in the disclosure are chosen, for example, from the addition salts with sodium hydroxide, potassium hydroxide, ammonia, amines and alkanolamines.

The dye composition in accordance with the disclosure may also contain at least one direct dye that may be chosen from neutral, acidic or cationic nitrobenzene dyes, neutral, acidic or cationic azo direct dyes, neutral, acidic or cationic quinone; for example, anthraquinone direct dyes, azine direct dyes, methine, azomethine, triarylmethane or indoamine direct dyes and natural direct dyes. These additional direct dyes may, for example, be chosen from cationic direct dyes and natural direct dyes.

Among the cationic direct dyes that may be used according to the disclosure, non-limiting mention may be made of the cationic azo direct dyes described in International Patent Application Nos. WO 95/15144 and WO 95/01772 and European Patent Application No. EP 714 954.

The cationic dyes may be chosen from:
1,3-dimethyl 2-[[4-(dimethylamino)phenyl]azo]-1H-imidazolium chloride,
1,3-dimethyl 2-[(4-aminophenyl)azo]-1H-imidazolium chloride,
1-methyl-4-[(methylphenylhydrazonomethyl]pyridinium methyl sulfate.

Among the natural direct dyes that may be used according to the disclosure, non-limiting mention may be made of lawsone, juglone, alizarin, purpurin, carminic acid, kermesic acid, purpurogallin, protocatechaldehyde, indigo, isatin, curcumin, spinulosin and apigenidin. Extracts or decoctions comprising these natural dyes, for example, henna-based poultices or extracts, may also be used.

The direct dye may, for example, be present in an amount ranging from 0.001% to 20% by weight, for example, from 0.005% to 10% by weight, relative to the total weight of the ready-to-use composition.

The dye composition in accordance with the disclosure may optionally contain at least one adjuvant conventionally used in compositions for dyeing the hair, such as antioxidants, penetrating agents, sequestering agents, fragrances, buffers, dispersants, surfactants other than those of the disclosure, conditioners such as, for example, volatile or non-volatile, modified or unmodified silicones, cationic polymers, cations, film-forming agents, ceramides, preserving agents, opacifiers, vitamins, provitamins and thickening polymers.

The at least one adjuvant may be present in an amount ranging from 0.01% to 20% by weight, relative to the weight of the composition.

Needless to say, the person skilled in the art will take care to select the at least one adjuvant wherein the advantageous properties intrinsically associated with the oxidation dye composition in accordance with the disclosure are not, or are not substantially, adversely affected by the adjuvant.

The medium that is suitable for dyeing, also known as the dye support, may for example, be comprised of water or of a mixture of water and at least one organic solvent to dissolve the compounds that would not be sufficiently water-soluble. As appropriate, this solvent may be a substrate of the enzyme such as ethanol or isopropanol. It may also be a non-substrate compound of the enzyme such as polyol ethers, for instance 2-butoxyethanol, propylene glycol, propylene glycol monomethyl ether and diethylene glycol monomethyl ether and monoethyl ether or phenoxyethanol, and mixtures thereof.

The solvents may be present in proportions ranging from, for example, 1% to 40% by weight, relative to the total weight of the dye composition, and further, for example, from 5% to 30% by weight, relative to the total weight of the dye composition.

The pH of the dye composition in accordance with the disclosure may range, for example, from 6 to 11, and further, for example, from 7 to 10. It may be adjusted for the desired value using acidifying or basifying agents usually used in the dyeing of keratin fibers, or using standard buffer systems.

Among the acidifying agents that may be mentioned, for example, are mineral or organic acids, for instance hydrochloric acid, orthophosphoric acid, sulphuric acid, carboxylic acids, for instance acetic acid, tartaric acid, citric acid and lactic acid, and sulphonic acids.

Among the basifying agents that may be mentioned, for example, are aqueous ammonia, alkyl metal carbonates, alkanolamines such as monoethanolamine, diethanolamine and triethanolamine and also derivatives thereof, sodium hydroxide, potassium hydroxide and the compounds of formula (IV): 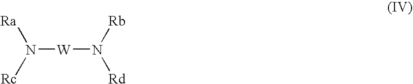
wherein:
W is a propylene residue optionally substituted with a group chosen from a hydroxyl group and a C₁-C₄ alkyl group;
Ra, Rb, Rc and Rd, which may be identical or different, are chosen from a hydrogen atom, C₁-C₄ alkyl groups and C₁-C₄ hydroxyalkyl groups.

The dye composition may be in various forms, such as in the form of thickened liquids, creams or gels, or in any other form that is suitable for dyeing keratin fibers, for example, human hair.

When the oxidation dyes and the alcohol oxidase are present in the same ready-to-use composition, said composition should be free of oxygen gas to avoid any premature oxidation of the oxidation dye(s).

Another aspect of the disclosure is also a process for dyeing keratin fibers, for example, human keratin fibers such as the hair, wherein at least one dye composition according to the disclosure is applied to these fibers, the duration of this application for a period that is sufficient to develop the desired coloration.

The color is then developed by bringing together the alcohol oxidase enzyme and its substrate in the presence of oxygen.

The composition is applied to the keratin fibers. After leaving it to act for a time ranging from 3 to 60 minutes, for example, from 5 to 40 minutes, the keratin fibers are rinsed, washed with shampoo, rinsed again and then dried.

When the dye composition is a composition in ready-to-use form, it comprises, in a medium that is suitable for dyeing keratin fibers, at least one oxidation dye precursor, at least one alcohol oxidase enzyme, at least one substrate for said enzyme and at least one nonionic surfactant, and the mixture is then stored in anaerobic form, free of oxygen gas.

According to one variant, the process comprises:
(1) separately storing a composition (A) comprising in a medium that is suitable for dyeing keratin fibers, at least one oxidation dye precursor, and a composition (B) comprising, in a medium that is suitable for dyeing keratin fibers, at least one alcohol oxidase enzyme, the composition (A) and/or the composition (B) comprising at least one substrate for said enzyme, and the composition (A) and/or the composition (B) comprising at least one sugar-based nonionic surfactant, and
(2) mixing together the compositions (A) and (B) at the time of use before applying this mixture to the keratin fibers.

According to one variant, the process comprises:
(1) separately storing a composition (A) comprising in a medium that is suitable for dyeing keratin fibers, at least one oxidation dye precursor, at least one substrate for said enzyme, and at least one sugar-based nonionic surfactant and a composition (B) comprising, in a medium that is suitable for dyeing keratin fibers, at least one alcohol oxidase enzyme, and
(2) mixing together the compositions (A) and (B) at the time of use before applying this mixture to the keratin fibers.

The colour may be revealed at acidic, neutral or alkaline pH. In the case where the process is performed using a composition (A) comprising at least one oxidation dye precursor, at least one substrate for said enzyme and at least one sugar-based nonionic surfactant and a composition (B) comprising at least one alcohol oxidase enzyme, the enzyme may be added to the composition of the disclosure just at the time of use, or it may be used starting with a composition comprising it, applied simultaneously or sequentially to the composition of the disclosure.

This composition (so-called oxidizing composition) may also contain various adjuvants conventionally used in compositions for dyeing the hair and as defined above.

The pH of the composition (B), so-called oxidizing composition, is such that, after mixing with the dye composition A, the pH of the resultant composition applied to the keratin fibers may, for example, range from 6 to 11, for example, from 7 to 10. It may be adjusted to the desired value by means of acidifying or basifying agents usually used in the dyeing of keratin fibers and as defined above.

The application of the composition according to the disclosure may, for example, be carried out at a temperature ranging from ambient temperature to 220° C., for example, from ambient temperature to 60° C.

Another aspect of the disclosure is a multi-compartment device or dyeing “kit”, wherein a first compartment contains the composition (A) as defined above and a second compartment contains the composition (B) as defined above. This device may be equipped with a means for applying the desired mixture to the hair, such as the devices described in French Patent No. FR-2 586 913 in the name of the Inventor.

Other than in the operating examples, and where otherwise indicated, all numbers expressing quantities of ingredients, reaction conditions, and so forth used in the specification and claims are to be understood as modified in all instances by the term “about.” Accordingly, unless indicated to the contrary, the numerical parameters set forth in this specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The following examples are intended to illustrate the invention in a non-limiting manner.

### EXAMPLE 1

The following composition was prepared:

| Constituent | Composition 1 |
|---|---|
| Alkylpolyglucoside | 8 g |
| (Oramix CG110 - SEPPIC) | |
| Ethanol | 25 g |
| para-Phenylenediamine | 3 × 10⁻³ mol |
| meta-Aminophenol | 3 × 10⁻³ mol |
| Alcohol oxidase | 20000 units |
| 2-Amino-2-methyl-1-propanol | qs pH 7 |
| Distilled water | qs 100 g |

The alcohol oxidase used was the enzyme sold by the company Biozyme Laboratories in liquid form at a concentration of 1980 units/ml.

The unit U corresponds to the amount of enzyme leading to the generation of 1 μmol of hydrogen peroxide per minute at pH 7.5 (100 mM phosphate buffer) and at a temperature of 25° C.

The above compositions were applied to locks of natural grey permanent-waved hair containing 90% white hairs, and left to act for 30 minutes. The bath ratio was set at 5. The alcohol oxidase was added extemporaneously. The hair was then rinsed, washed with a standard shampoo and then dried.

A fast and homogeneous coloration in khaki-green shades was obtained.

1. A composition for dyeing keratin fibers comprising, in a medium that is suitable for dyeing,
at least one oxidation dye precursor,
at least one alcohol oxidase enzyme,
at least one substrate for the at least one alcohol oxidase enzyme, and
at least one sugar-based nonionic surfactant,
wherein
the at least one substrate for the at least one alcohol oxidase enzyme is optionally substituted totally or partially by an oxidation dye precursor in the case where the at least one substrate for the at least one alcohol oxidase enzyme bears at least one aromatic or aliphatic alcohol function.

2. A composition according to claim 1, wherein the at least one sugar-based nonionic surfactant is chosen from alkylpolyglucosides; fatty acid esters of a sugar or of an alkyl-sugar; fatty sucramides; and mixtures thereof.

3. A composition according to claim 2, wherein the at least one sugar-based nonionic surfactant is an alkylglucoside of formula (I):
R₁—O—(R₂—O)ₐ—(L)_{b} (I)
wherein:
R¹ is chosen from linear and branched alkyl and alkenyl groups comprising from 8 to 24 carbon atoms and alkylphenyl groups wherein the linear and branched alkyl group comprises from 8 to 24 carbon atoms;
R² is chosen from alkylene groups comprising from 2 to 4 carbon atoms;
L is a reduced sugar comprising 5 to 6 carbon atoms;
a is a number ranging from 0 to 10; and
b is a number ranging from 1 to 15.

4. A composition according to claim 3, wherein the at least one sugar-based nonionic surfactant is an alkylglucoside of formula (I) wherein:
R¹ is chosen from linear and branched alkyl and alkenyl groups comprising from 9 to 14 carbon atoms;
a is a number ranging from 0 to 3;
b is a number ranging from 1 to 15; and
L is chosen from glucose, fructose and galactose.

5. A composition according to claim 2, wherein the at least one sugar-based nonionic surfactant is chosen from (C₁-C₄)alkylglucoside esters, glucose esters and sucrose monoesters.

6. A composition according to claim 5, wherein the at least one sugar-based nonionic surfactant is a (C₁-C₄)alkylglucoside ester chosen from methylglucoside monostearate, methylglucoside sesquistearate, ethyl-6-glucoside decanoate, the mixture (82/7) of ethyl-6-glucoside mono- and dicocoate, the mixture (84/8) of ethyl-6-glucoside mono- and dilaurate, and C₁₂-C₁₈ fatty acid monoesters of butylglucoside.

7. A composition according to claim 5, wherein the at least one sugar-based nonionic surfactant is a glucose ester chosen from O-hexadecanoyl-6-αD-glucose, O-octanoyl-6-D-glucose, O-oleyl-6-D-glucose and O-linoleyl-6-D-glucose.

8. A composition according to claim 5, wherein the at least one sugar-based nonionic surfactant is a sucrose monoester chosen from sucrose monolaurate and sucrose monococoate.

9. A composition according to claim 2, wherein the at least one sugar-based nonionic surfactant is a fatty sucramide chosen from N-substituted aldonamides, polyhydroxylated fatty acid amides, and mixtures thereof.

10. A composition according to claim 9, wherein the at least one sugar-based nonionic surfactant is an N-substituted aldonamide chosen from N-substituted lactobionamides, N-substituted maltobionamides, N-substituted cellobionamides, N-substituted mellibionamides and N-substituted gentiobionamides.

11. A composition according to claim 10, wherein the at least one sugar-based nonionic surfactant is an N-substituted aldonamide chosen from:
N-alkyl lactobionamides, N-alkyl maltobionamides, N-alkyl cellobionamides, N-alkylmellibionamides and N-alkyl gentiobionamides, each of which are optionally mono- or disubstituted with groups chosen from linear and branched, saturated and unsaturated aliphatic hydrocarbon-based groups, optionally comprising hetero atoms;
N-lactobionyl amino acid esters, N-maltobionyl amino acid esters, N-mellibionyl amino acid esters, N-cellobionyl amino acid esters and N-gentiobionyl amino acid esters,wherein the amino acid is chosen from the group formed by alanine, valine, glycine, lysine, leucine, arginine, aspartic acid, glutamic acid, threonine, serine, cysteine, histidine, tyrosine, methionine, β-alanine, sarcosine, γ-aminobutyric acid, ornithine, citrulline, and equivalents thereof; said N-lactobionyl amino acid esters is monosubstituted with a group of formula (II): 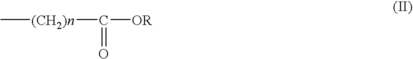
wherein:
R is an aliphatic hydrocarbon-based group comprising from 1 to 36 carbon atoms; and
n is an integer greater than 1;
N-(alkyloxy)alkyl-lactobionamides mono- or disubstituted with a group —(CH₂)ₙOR′, wherein R′ is chosen from aliphatic hydrocarbon-based groups, aromatic hydrocarbon-based groups and cycloaliphatic hydrocarbon-based groups; and
N-(polyalkyloxy)alkyl lactobionamides, N-(polyalkyloxy)alkyl maltobionamides, N-(polyalkyloxy)alkyl cellobionamides, N-(polyalkyloxy)alkyl mellibionamides and N-(polyalkyloxy)alkyl gentiobionamides, each of which are mono- and disubstituted with a group —R₁—(OR₁)ₙR₁R₂ wherein
R₁ is chosen from alkylene groups and mixtures thereof;
n is an integer greater than 1; and
R₂ is chosen from lactobionamide, maltobionamide, cellobionamide, melliobionamide and gentiobionamide groups.

12. A composition according to claim 9, wherein the at least one sugar-based nonionic surfactant is chosen from a polyhydroxylated fatty amide of formula (III): 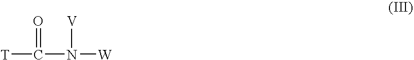
wherein:
T is chosen from C₅-C₃, hydrocarbon-based groups and mixtures thereof;
V is chosen from a hydrogen atom, C₁-C₄ hydrocarbon-based groups, 2-hydroxyethyl, 2-hydroxypropyl, and mixtures thereof;
W is chosen from polyhydroxy hydrocarbon-based groups with a linear hydrocarbon-based chain comprising at least 3 hydroxyl groups directly linked to the chain, and an alkoxylated derivative of a polyhydroxy hydrocarbon-based group.

13. A composition according to claim 12, wherein the at least one sugar-based nonionic surfactant is a polyhydroxylated fatty amide of formula (III) wherein W is a reducing sugar derivative chosen from glucose, maltose, lactose, galactose, mannose and xylose.

14. A composition according to claim 13, wherein the at least one sugar-based nonionic surfactant is a polyhydroxylated fatty amide of formula (III) wherein W is chosen from:
—CH₂—(CHOH)ₙ—CH₂OH;
—CH—(CH₂OH)—(CHOH)ₙ₋₁—CH₂O H; and
—CH₂—(CHOH)₂(CHOR′)(CHOH)—CH₂OH
wherein:
n is an integer ranging from 3 to 5; and
R′ is chosen from hydrogen atom, cyclic and aliphatic monosaccharides and alkoxylated derivatives thereof.

15. A composition according to claim 13, wherein the at least one sugar-based nonionic surfactant is a polyhydroxylated fatty amide of formula (III) wherein the group T-CON=is chosen from cocamide, stearamide, oleamide, lauramide, myristyramide, capricamide, palmitamide and tallow amide.

16. A composition according to claim 1, wherein the at least one sugar-based nonionic surfactant is present in an amount ranging from 0.05% to 30% by weight, relative to the total weight of the composition.

17. A composition according to claim 1, wherein the at least one alcohol oxidase enzyme is chosen from primary alcohol oxidases (EC1.1.3.13), secondary alcohol oxidases (EC 1.1.3.18), long-hydrocarbon-chain alcohol oxidases (EC 1.1.3.20), polyvinyl alcohol oxidases (EC 1.1.3.30), vanillyl alcohol oxidase (EC 1.1.3.38) and aromatic alcohol oxidases (EC 1.1.3.7).

18. A composition according to claim 17, wherein the at least one alcohol oxidase enzyme is chosen from enzymes derived from one of the following species: Rhodococcus erythropolis, Pseudomonas pseudoalcaligenes, Aspergillus niger, Kamagataella pastoris, Phanerochaete chrysosporium, Polyporus obtusus, Hansenula polymorpha, Poria contigua, Penicillium simplicissimum, Pleurotus pulmonarius, Pichia sp. (pastoris, methanolica, angusta) and Candida sp. (boidinii, albicans, tropicalis), Pinus strobus, Gastropode mollusc, and Manduca sexta.

19. A composition according to claim 17, wherein the concentration of the at least one alcohol oxidase enzyme is present in an amount ranging from 0.05 to 20% by weight, relative to the total weight of the composition.

20. A composition according to claim 1, wherein the at least one substrate for the at least one alcohol oxidase enzyme is chosen from primary alcohols, secondary alcohols, long-hydrocarbon-chain alcohols and aromatic alcohols.

21. A composition according to claim 20, wherein the at least one substrate for the at least one alcohol oxidase enzyme is present in an amount ranging from 0.01% to 60% by weight, relative to the total weight of the composition.

22. A composition according to claim 1, wherein the at least one oxidation dye precursor is a standard oxidation base chosen from para-phenylenediamines, bis(phenyl)alkylenediamines, para-aminophenols, ortho-aminophenols and heterocyclic bases, and the addition salts thereof.

23. A composition according to claim 22, wherein the standard oxidation base is present in an amount ranging from 0.0001% to 20% by weight, relative to the total weight of the composition.

24. A composition according to claim 1, wherein the at least one oxidation dye precursor is a standard oxidation coupler chosen from meta-phenylenediamines, meta-aminophenols, meta-diphenols, naphthalenic couplers and heterocyclic couplers, and the addition salts thereof.

25. A composition according to claim 24, wherein the standard oxidation coupler is present in an amount ranging from 0.0001% to 20% by weight, relative to the total weight of the composition.

26. A composition according to claim 1, wherein the composition comprises at least one dye chosen from cationic dyes and natural direct dyes.

27. A process for dyeing keratin fibers comprising applying to the fibers for a period that is sufficient to develop the desired coloration at least one composition for dyeing keratin fibers comprising, in a medium that is suitable for dyeing,
at least one oxidation dye precursor,
at least one alcohol oxidase enzyme,
at least one substrate for the at least one alcohol oxidase enzyme, and
at least one sugar-based nonionic surfactant,
wherein
the at least one substrate for the at least one alcohol oxidase enzyme is optionally substituted totally or partially by an oxidation dye precursor in the case where the substrate for the at least one alcohol oxidase enzyme bears at least one aromatic or aliphatic alcohol function.

28. A process according to claim 27, wherein the composition is a ready-to-use composition comprising, in a medium that is suitable for dyeing keratin fibers,
at least one oxidation dye precursor,
at least one alcohol oxidase enzyme,
at least one substrate for the at least one alcohol oxidase enzyme, and
at least one sugar-based nonionic surfactant,
wherein
the at least one substrate for the at least one alcohol oxidase enzyme is optionally substituted totally or partially by an oxidation dye precursor in the case where the substrate for the at least one alcohol oxidase enzyme bears at least one aromatic or aliphatic alcohol function, and wherein the mixture is stored in anaerobic form, free of oxygen gas.

29. A process according to claim 28, comprising:
(1) separately storing a composition (A) comprising in a medium that is suitable for dyeing, at least one oxidation dye precursor, and a composition (B) comprising, in a medium that is suitable for keratin fibers, at least one alcohol oxidase enzyme, the composition (A) and/or the composition (B) comprising at least one substrate for said at least one alcohol oxidase enzyme and the composition (A) and/or the composition (B) comprising at least one sugar-based nonionic surfactant, and
(2) mixing together the compositions (A) and (B) at the time of use before applying this mixture to the keratin fibers.

30. A process according to claim 29, comprising
(1) separately storing a composition (A) comprising, in a medium that is suitable for dyeing, at least one oxidation dye precursor, at least one substrate for the alcohol oxidase enzyme and at least one sugar-based nonionic surfactant and a composition (B) comprising, in a medium that is suitable for keratin fibers, at least one alcohol oxidase enzyme, and
(2) mixing together the compositions (A) and (B) at the time of use before applying this mixture to the keratin fibers.

31. A multi-compartment device or dyeing “kit”, comprising a first compartment comprising the composition (A) and a second compartment comprising the composition (B) as defined in claim 29.
